(19) United States
(12) Patent Application Publication
(10) Pub. No.: US 2025/0246306 A1
(43) Pub. Date: Jul. 31, 2025

(54) METHOD FOR PROVIDING INFORMATION ON HYPOTENSION AND DEVICE USING THE SAME

(71) Applicant: INDUSTRY-ACADEMIC COOPERATION FOUNDATION, YONSEI UNIVERSITY, Seoul (KR)

(72) Inventors: Duk Yong Yoon, Suwon-si (KR); Ji Hoon Seo, Gwangmyeong-si (KR); Chan Min Park, Yongin-si (KR); Chang Ho Han, Yongin-si (KR)

(73) Assignee: INDUSTRY-ACADEMIC COOPERATION FOUNDATION, YONSEI UNIVERSITY, Seoul (KR)

(21) Appl. No.: 19/007,290

(22) Filed: Dec. 31, 2024

(30) Foreign Application Priority Data
Jan. 26, 2024 (KR) 10-2024-0012559

(51) Int. Cl. G16H 50/20; G16H 10/60

(52) U.S. Cl. CPC G16H 50/20; G16H 10/60

(57) ABSTRACT

According to this specification, a method for providing information on hypotension implemented by a processor includes: receiving first clinical data and second clinical data for an individual; extracting a statistical value for the received first clinical data as a first feature; extracting an embedding vector for the received second clinical data as a second feature; and predicting whether the hypotension occurs in the individual within a first period by inputting the first feature and the second feature to a prediction model trained to predict whether the hypotension occurs using the first feature and the second feature as inputs.

### CROSS-REFERENCE TO RELATED APPLICATIONS

This application claims the priority of Korean Patent Application No. 10-2024-0012559 filed on Jan. 26, 2024, in the Korean Intellectual Property Office, the disclosure of which is incorporated herein by reference.

### BACKGROUND

### Field

The present disclosure relates to a device for providing information on hypotension and a kit using the same.

### Description of the Related Art

An intensive care unit is a place where close monitoring of patients with unstable vital signs and immediate resuscitation treatment according to changes in their condition is crucial. Recently, as the patient population is aging and the number of patients with chronic diseases is increasing due to the development of medical technology and industrial technology, duration of stay in the intensive care unit tends to be longer. Such long-term illness in the intensive care unit causes various complications such as weakening of underlying disease and deterioration in physical function, which may increase the severity and mortality rate in general wards and affect medical costs. In addition, the long-term illness may lower the quality of medical care and limit the efficient use of limited medical resources. Therefore, when characteristics of patients in the intensive care unit can be identified and factors affecting prognosis may be predicted in advance, a direction of patient treatment may be quickly determined, which may not only have a positive effect on the prognosis of the patients, but also improve the overall quality of the medical care.

In this regard, the incidence of hypotension in patients admitted to the intensive care unit is 47% to 72%, and the hypotension is one of the common symptoms in patients in the intensive care unit. The hypotension is a major factor in the development of distributive shock, cardiogenic shock, hypovolemic shock, and obstructive shock that lead to major organ failure and death. Furthermore, the hypotension is a chronic risk factor associated with increased acute kidney injury, myocardial ischemia, and a mortality rate. Therefore, early detection and prompt management of the hypotension are very important for improving the prognosis of patients.

Currently, the hypotension is monitored through hemodynamic variables in the intensive care unit, and patients may be identified through various early warning scores (APACHE II, SAPS II, SOFA). However, most of these approaches are calculated manually and do not provide real-time feedback on the patient's conditions that change in real time. Therefore, the current manual early warning system has low reliability and has limitations in its application in actual clinical settings. Therefore, there is a continuous demand for the development of a method that may predict the hypotension early.

The technology that forms the background of the invention has been written to facilitate a better understanding of the present disclosure. It should not be understood that the matters described in the technology that form the background of the invention exist as the related art.

### SUMMARY

A technology of measuring hypotension, that is, blood pressure may be classified into invasive or noninvasive methods. As a standard noninvasive blood pressure (NIBP) measurement technique, a measuring tape-based oscilloscope method is applied. This approach produces individual systolic and diastolic blood pressure results. This method does not have a significant risk, but is not easy to accurately measure blood pressure and has low reliability for clinical use. In the invasive blood pressure (IBP) monitoring, a needle-type blood pressure sensor is inserted into a patient's artery to continuously and accurately measure the blood pressure, but this approach involves the risk of infection and bleeding and also causes pain to the patient.

In this regard, the inventors of the present disclosure focused on an early prediction model of hypotension using machine learning. Recently, research on early models using machine learning has been actively conducted. More specifically, the study by Fournier et al., in 2009 aimed to predict hypotension using vital sign and ECG data from 60 patients, but showed a low accuracy of 0.80, and the study by Lee et al., in 2011 attempted to predict hypotension from 1 to 4 hours using vital sign data of 30 minutes, but had low accuracy and a very low positive predictive value (PPV) of 0.159. In addition, the study by Angelotti et al., in 2018 combined vital sign data and ECG data and set a prediction window of 10 minutes with data for 20 minutes to predict hypotension, but had a low AUC of 0.68, and the study by Ribeiro et al., in 2021 predicted hypotension by setting a prediction window of 1 hour using vital sign data for 1 hour, but had low accuracy of 75.9% similar to other conventional studies. That is, all the conventional hypotension prediction studies using machine learning showed low performance, and have been difficult to apply practically in clinical settings.

Meanwhile, the inventors of the present disclosure recognized the problem that it was difficult to provide sufficient response time in clinical settings when using a prediction window of about 30 minutes in the conventional studies. Accordingly, the inventors of the present disclosure found that when the prediction window was extended to 60 minutes, the time limitations in the conventional studies were overcome. In addition, the inventors of the present disclosure found that when waveform data was used together with vital sign data, the accuracy of prediction was improved even without using invasive indicators.

In the end, the inventors of the present disclosure have improved the ability to secure sufficient preparation time before the occurrence of hypotension in actual clinical settings by extending the prediction window, and derived high predictive performance by additionally utilizing factors with high predictive power among various factors causing hypotension in addition to the conventional predictive indicators.

An object to be achieved by the present disclosure is to provide a method for providing information on hypotension capable of predicting the occurrence of hypotension with high accuracy by inputting first clinical data for a vital sign and second clinical data for waveform data to a prediction model.

Another object to be achieved by the present disclosure is to provide a device for providing information on hypotension based on the above-described method for providing information on hypotension.

Aspects of the present disclosure are not limited to the above-mentioned aspects. That is, other aspects that are not mentioned may be obviously understood by those skilled in the art from the following specification.

A method for providing information on hypotension implemented by a processor according to an exemplary embodiment of the present disclosure includes: receiving first clinical data and second clinical data for an individual; extracting a statistical value for the received first clinical data as a first feature; extracting an embedding vector for the received second clinical data as a second feature; and predicting whether the hypotension occurs in the individual within a first period by inputting the first feature and the second feature to a prediction model trained to predict whether the hypotension occurs using the first feature and the second feature as inputs.

The hypotension may mean that a systolic blood pressure (SBP) of 90 mmHg or less and a mean arterial pressure (MAP) of 60 mmHg or less occur 5 times or more for about 10 minutes.

The first clinical data may include at least one of heart rate, respiration (RESP), O2 saturation (SpO2), and non-invasive blood pressure (NBP), but is not limited thereto.

The second clinical data may include at least one of ECG lead II and photoplethysmography (PPG), but is not limited thereto.

The statistical value may include at least one of mean, variance, median, a min value, a max value, quantile, and an exponentially weighted moving average, but is not limited thereto.

The extracting of the statistical value as the first feature may include extracting as the first feature a first window having a size smaller than that of a first period from the received first clinical data by the time-series interval movement (rolling windows) of the first windows at a predetermined time interval.

The extracting of the embedding vector as the second feature may include extracting as the second feature the first window having the size smaller than that of the first period from the received second clinical data by the rolling windows of the first window at the predetermined time interval.

The extracting of the embedding vector as the second feature may include: extracting first extraction data having a size of the first window based on the first window from the received second clinical data; segmenting the extracted first extraction data into a predetermined size; extracting second extraction data for a portion of an initial period from the segmented first extraction data; sampling the extracted second extraction data so that the extracted second extraction data is transformed into an embedding feature in a digital form; and inputting the embedding feature to a transformer encoder and outputting the embedding feature as the embedding vector.

The sampling step may further include transforming the second extraction data into a two-dimensional or more embedding feature by inputting the extracted second extraction data to a transformation model trained to transform the second extraction data into the two-dimensional or more embedding feature using the second extraction data as input.

The method may further include: prior to the extracting, selecting a hypotension group from the received first clinical data and second clinical data; setting a first prediction window of the first period in the first clinical data of the selected hypotension group; setting a second prediction window of the first period in the second clinical data of the selected hypotension group; extracting a first learning feature and a second learning feature from each of the set first prediction window and second prediction window by the rolling windows of a first window having a size smaller than that of the first period at a predetermined time interval; and training the prediction model based on the extracted first learning feature and second learning feature.

The setting of the first prediction window may include: setting a first event time for a first hypotension occurrence event time in the first clinical data of the selected hypotension group; setting a second event time before the first period from the first event time; and setting the first clinical data for the hypotension group from the second event time to the first event time as the first prediction window.

The setting of the second prediction window may include: setting an event time corresponding to the first event time in the second clinical data of the selected hypotension group as a third event time; setting a fourth event time before the first period from the third event time; and setting the second clinical data for a non-hypotension group from the fourth event time to the third event time as the second prediction window.

The method may further include: prior to the extracting, selecting a non-hypotension group from the received first clinical data and second clinical data; setting a third prediction window of the first period in the first clinical data of the selected non-hypotension group; setting a fourth prediction window of the first period in the second clinical data of the selected non-hypotension group; extracting a third learning feature and a fourth learning feature from each of the set third prediction window and fourth prediction window by the rolling windows of a first window having a size smaller than that of the first period at a predetermined time interval; and training the prediction model based on the extracted third learning feature and fourth learning feature.

The setting of the third prediction window may include: setting a first event time for a first hypotension occurrence event time in the first clinical data of the selected non-hypotension group; setting a second event time before the first period from the first event time; and setting the first clinical data for the non-hypotension group from the second event time to the first event time as the third prediction window.

The setting of the fourth prediction window may include: setting an event time corresponding to the first event time in the second clinical data of the selected non-hypotension group as a third event time; setting a fourth event time before the first period from the third event time; and setting the second clinical data for the non-hypotension group from the fourth event time to the third event time as the fourth prediction window.

A device for providing information on hypotension according to another exemplary embodiment of the present disclosure includes: a communication unit configured to receive first clinical data and second clinical data for an individual; and a processor configured to communicate with the communication unit, in which the processor may be configured to extract a statistical value for the received first clinical data as a first feature; extract an embedding vector for the received second clinical data as a second feature; and predict whether the hypotension occurs in the individual within a first period by inputting the first feature and the second feature to a prediction model trained to predict whether the hypotension occurs using the first feature and the second feature as inputs.

The hypotension may mean that a systolic blood pressure (SBP) of 90 mmHg or less and a mean arterial pressure (MAP) of 60 mmHg or less occur 5 times or more for about 10 minutes.

The first clinical data may include at least one of heart rate, respiration (RESP), O2 saturation (SpO2), and non-invasive blood pressure (NBP), but is not limited thereto.

The second clinical data may include at least one of ECG lead II and photoplethysmography (PPG), but is not limited thereto.

The statistical value may include at least one of mean, variance, median, a min value, a max value, quantile, and an exponentially weighted moving average, but is not limited thereto.

The processor may be configured to extract as the first feature a first window having a size smaller than that of a first period from the received first clinical data by the rolling windows of the first window at the predetermined time interval.

The processor may be configured to extract as the second feature the first window having a size smaller than that of the first period from the received second clinical data by the rolling windows of the first window at the predetermined time interval.

The processor may extract first extraction data having a size of the first window based on the first window from the received second clinical data, segment the extracted first extraction data into a predetermined size, extract second extraction data for a portion of an initial period from the segmented first extraction data, sample the extracted second extraction data so that the extracted second extraction data is transformed into an embedding feature in a digital form, and input the embedding feature to a transformer encoder and output the embedding feature as the embedding vector.

The processor may be further configured to transform the second extraction data into a two-dimensional or more embedding feature by inputting the extracted second extraction data to a transformation model trained to transform the second extraction data into the two-dimensional or more embedding feature using the second extraction data as input.

The processor may be further configured to select a hypotension group from the received first clinical data and second clinical data, set a first prediction window of the first period in the first clinical data of the selected hypotension group, set a second prediction window of the first period in the second clinical data of the selected hypotension group, extract a first learning feature and a second learning feature from each of the set first prediction window and second prediction window by the rolling windows of a first window having a size smaller than that of the first period at a predetermined time interval, and train the prediction model based on the extracted first learning feature and second learning feature.

The processor may be configured to set a first event time for a first hypotension occurrence event time in the first clinical data of the selected hypotension group, set a second event time before the first period from the first event time, and set the first clinical data for the hypotension group from the second event time to the first event time as the first prediction window.

The processor may be configured to set an event time corresponding to the first event time in the second clinical data of the selected hypotension group as a third event time, set a fourth event time before the first period from the third event time, and set the second clinical data for a non-hypotension group from the fourth event time to the third event time as the second prediction window.

The processor may be further configured to select a non-hypotension group from the received first clinical data and second clinical data, set a third prediction window of the first period in the first clinical data of the selected non-hypotension group, set a fourth prediction window of the first period in the second clinical data of the selected non-hypotension group, extract a third learning feature and a fourth learning feature from each of the set third prediction window and fourth prediction window by the rolling windows of a first window having a size smaller than that of the first period at a predetermined time interval, and train the prediction model based on the extracted third learning feature and fourth learning feature.

The processor may be configured to set a first event time for a first hypotension occurrence event time in the first clinical data of the selected non-hypotension group, set a second event time before the first period from the first event time, and set the first clinical data for the non-hypotension group from the second event time to the first event time as the third prediction window.

The processor may be configured to set an event time corresponding to the first event time in the second clinical data of the selected non-hypotension group as a third event time, set a fourth event time before the first period from the third event time, and set the second clinical data for a non-hypotension group from the fourth event time to the third event time as the fourth prediction window.

Hereinafter, the present disclosure will be described in more detail with reference to exemplary embodiments. However, since these exemplary embodiments are only intended to exemplarily describe the present disclosure, the scope of the present disclosure should not be construed as being limited by these exemplary embodiments.

According to the present disclosure, by predicting the occurrence of hypotension in an individual, the medical staff can quickly determine the diagnosis for the hypotension in the individual, so positive results can be expected on the prognosis for diseases caused by hypotension. In addition, as the unnecessary medical prescriptions and treatments are minimized, it is possible to reduce the temporal and cost burden on the individual (patient).

In addition, as the present disclosure is based on the non-invasive clinical data, the present disclosure can be applied to patients who have difficulty in inserting an arterial catheter.

In addition, as the present disclosure is based on the clinical data that can be measured continuously and in real time, it is possible to continuously and quickly predict the risk factors (hypotension) in advance, thereby reducing the time required for the conventional vital sign analysis and providing faster analysis results.

As a result, according to the present disclosure, it is possible to help secure the sufficient response time before the hypotension occurs in urgent situations such as emergency rooms, reduce the burden on patients for invasive treatment (arterial catheter), and improve versatility that can be applied to various patient groups. Accordingly, according to the present disclosure, it is possible to greatly contribute to preemptive response in urgent situations such as shock.

The effects according to the present disclosure are not limited to the contents exemplified above, and further various effects are included in the present specification.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1A is an exemplary diagram illustrating a system based on a device for providing information on hypotension according to an exemplary embodiment of the present disclosure;

FIG. 1B is a block diagram illustrating a configuration of the device for providing information on hypotension according to an exemplary embodiment of the present disclosure;

FIG. 1C is a block diagram illustrating a configuration of a medical staff device according to an exemplary embodiment of the present disclosure;

FIG. 1D is a schematic diagram illustrating an interface between a medical staff device and a vital sign monitoring device according to an exemplary embodiment of the present disclosure;

FIG. 2 is a schematic diagram illustrating a method for providing information on hypotension according to an exemplary embodiment of the present disclosure;

FIGS. 3A and 3B are schematic diagrams illustrating extraction of first feature and second feature in the method for providing information on hypotension according to an exemplary embodiment of the present disclosure;

FIGS. 4A and 4B are schematic diagrams illustrating the extraction of the learning feature in the method for providing information on hypotension according to an exemplary embodiment of the present disclosure;

FIG. 5 is a flowchart illustrating the method for providing information on hypotension according to an exemplary embodiment of the present disclosure;

FIG. 6A is a diagram illustrating an AUROC curve result for the method for providing information on hypotension according to an exemplary embodiment of the present disclosure;

FIG. 6B is a diagram illustrating an AUPRC curve result for the method for providing information on hypotension according to an exemplary embodiment of the present disclosure;

FIG. 7 is a diagram illustrating a performance evaluation result for a prediction model of the method for providing information on hypotension according to an exemplary embodiment of the present disclosure;

FIG. 8 is a diagram illustrating a SHAP value result for the method for providing information on hypotension according to an exemplary embodiment of the present disclosure; and

FIG. 9 is a consistency evaluation result for a method for providing information on hypotension according to an exemplary embodiment of the present disclosure.

### DETAILED DESCRIPTION OF THE EMBODIMENT

Advantages and features of the present disclosure and methods to achieve them will be elucidated from exemplary embodiments described below in detail with reference to the accompanying drawings. However, the present disclosure is not limited to exemplary embodiments disclosed below, and may be implemented in various different forms, these exemplary embodiments will be provided only in order to make the present disclosure complete and allow one of ordinary skill in the art to which the present disclosure pertains to completely recognize the scope of the present disclosure.

The term “or” used herein means “and/or” unless otherwise stated.

The term “about” used herein refers to a normal error range for each value that is readily known to those skilled in the art. References to “about” value or parameter herein include exemplary embodiments relating to that value or parameter itself. Furthermore, the term “about” refers to a range of values that fall within 10% in any one direction (exceeding or less than) of the mentioned reference value, unless otherwise stated or otherwise apparent from the context.

The term “patient or individual” used herein is used interchangeably and refers to any single animal, more preferably a mammal (including non-human animals such as cats, dogs, horses, rabbits, zoo animals, cattle, pigs, sheep, and non-human primates) for which treatment is desired. The patient referred to in various exemplary embodiments herein may be a human.

### System Based on Device for Providing Information on Hypotension According to Exemplary Embodiment of Present Disclosure

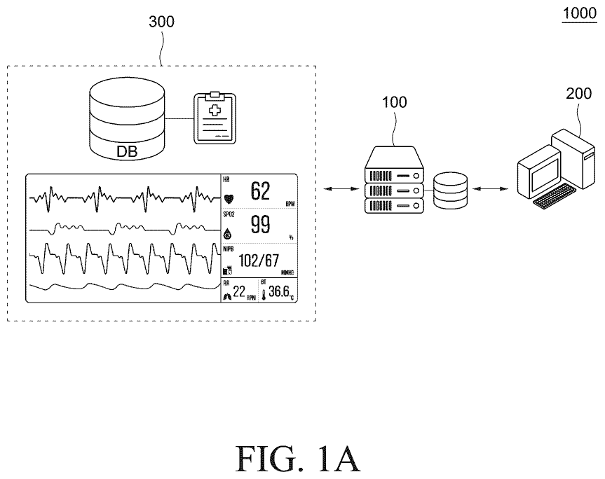

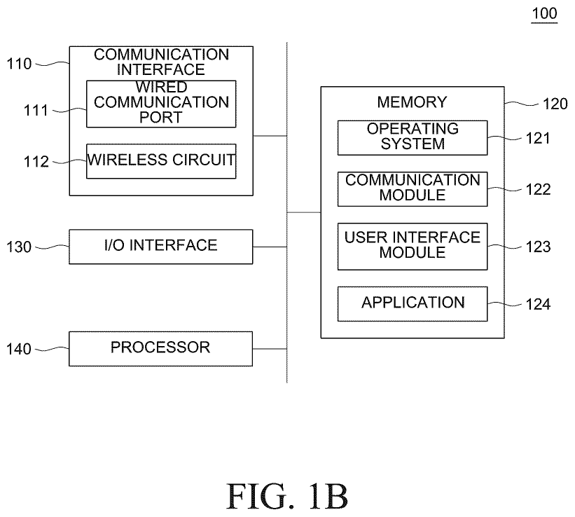

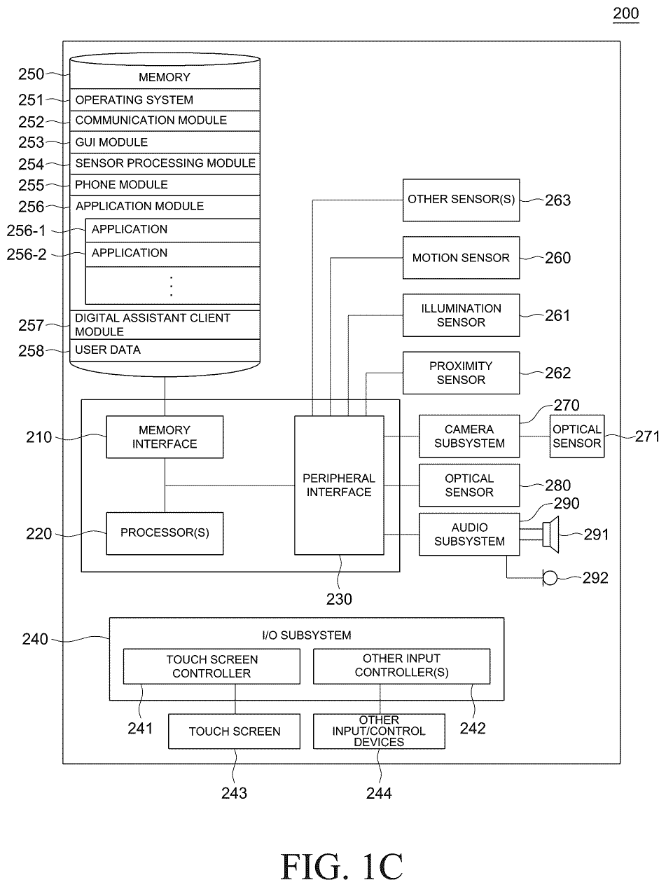

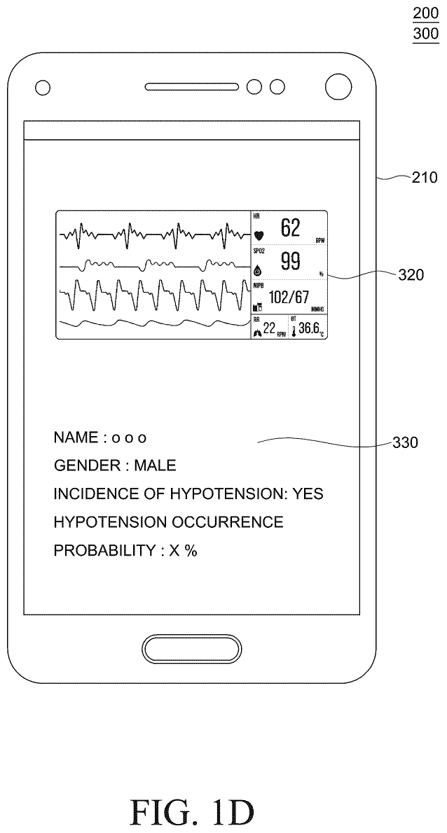

FIG. 1A is an exemplary diagram illustrating a system based on a device for providing information on hypotension according to an exemplary embodiment of the present disclosure.

First, referring to FIG. 1A, a system 1000 for providing information on hypotension may be a system configured to predict and provide information related to hypotension of an individual based on various clinical monitoring data collected from the individual.

In this case, the system 1000 for providing information on angiography may be configured to include a device 100 for providing information on hypotension that generates respective features based on clinical monitoring data collected from the individual, that is, first clinical data and second clinical data, and predict the hypotension of the individual based on the generated features, a medical staff device 200 that transmits and receives the information on the angiography, and a vital sign monitoring device 300.

In this case, the device 100 for providing information on hypotension, the medical staff device 200, and the vital sign monitoring device 300 may transmit and receive various types of information through wired and wireless communication.

More specifically, the device 100 for providing information on hypotension, the medical staff device 200, and the vital sign monitoring device 300 may communicate in a wired manner that is directly connected by a cable to perform transmission and reception, but may preferably communicate wirelessly to perform transmission and reception without a cable.

Therefore, the device 100 for providing information on hypotension, the medical staff device 200, and the vital sign monitoring device 300 may be connected to a network for wireless communication, and the network may be a closed network such as a local area network (LAN) and a wide area network (WAN), an open network such as the Internet, and a short-range wireless communication such as Bluetooth, near field communication (NFC), radio-frequency identification (RFID), Wi-Fi, and/or Zigbee, but is not limited thereto.

The device 100 for providing information on hypotension according to an exemplary embodiment of the present disclosure may include a general-purpose computer, a laptop, and/or a data server, etc., that generate a first feature for statistical values and a second feature for an embedding vector based on clinical data for an individual collected from the medical staff device 200 and/or the vital sign monitoring device 300, that is, first clinical data including heart rate (HR), respiration (RESP), O2 saturation (SpO2), and non-invasive blood pressure (NBP), and second clinical data including ECG lead II and photoplethysmography (PPG), and furthermore perform various operations for predicting the hypotension of the individual.

More specifically, FIG. 1B illustrates a block diagram illustrating a configuration of the device for providing information on hypotension according to an exemplary embodiment of the present disclosure. The device 100 for providing information on hypotension may include a communication interface 110, a memory 120, an I/O interface 113, and a processor 114, and each component may communicate with one another through one or more communication buses or signal lines.

The communication interface 110 may mean the communication unit 110, and may be connected to the medical staff device 200 and the vital sign monitoring device 300 via a wired/wireless communication network to transmit and receive data. For example, the communication interface 110 may receive various data (clinical data, extraction data, prediction data) about an individual in real time from the medical staff device 200 and/or the vital sign monitoring device 300. As another example, the communication interface 110 may transmit various medical data related to an individual from the medical staff device 200.

Meanwhile, the communication interface 110 that enables the transmission and reception of the data includes a wired communication port 111 and a wireless circuit 112, and the wired communication port 311 may include one or more wired interfaces such as Ethernet, a universal serial bus (USB), FireWire, etc. In addition, the wireless circuit 112 may transmit and receive data with an external device via RF signals or optical signals. In addition, the wireless communication may use at least one of a plurality of communication standards, protocols, and technologies, such as GSM, EDGE, CDMA, TDMA, Bluetooth, Wi-Fi, VoIP, Wi-MAX, or any other suitable communication protocol.

The memory 120 may be used in the device 100 for providing information on hypotension and may store various data derived and generated.

More specifically, the memory 120 may store a prediction model and may also store various data extracted or generated (predicted) from the above-described prediction model. For example, the memory 120 may include, but is not limited to, various algorithms, parameters, functions, extracted (generated) data, etc., included in the model.

Furthermore, the memory 120 may include all of various data related to the output data. For example, the memory 120 may include, but is not limited to, various extraction data (first feature, second feature, first learning feature, second learning feature, third learning feature, fourth learning feature) of the process for predicting hypotension of an individual.

In addition, the memory 120 may store various data, such as clinical data, collected from the medical staff device 200 or the vital sign monitoring device 300.

More specifically, the clinical data may include a non-invasive vital sign of an individual, i.e., clinical monitoring data derived (generated) from the vital sign monitoring device. For example, the clinical data may include, but is not limited to, the first clinical data including the heart rate (HR), the respiration (RESP), the O2 saturation (SpO2), and the non-invasive blood pressure (NBP), and the second clinical data including the ECG lead II and the photoplethysmography (PPG).

In various exemplary embodiments, the memory 120 may include volatile or nonvolatile recording media capable of storing various data, commands, and information. For example, the memory 120 may include at least one type of storage media such as flash memory type, hard disk type, multimedia card micro type, card type memory (e.g., SD or XD memory, etc.), RAM, SRAM, ROM, EEPROM, PROM, network storage, cloud, and blockchain database.

In various exemplary embodiments, the memory 120 may store at least one configuration of an operating system 121, a communication module 122, a user interface module 123, and one or more applications 124.

The operating system 121 (e.g., embedded operating system such as LINUX, UNIX, MAC OS, WINDOWS, and VxWorks) may include various software components and drivers for controlling and managing general system operations (e.g., memory management, storage device control, power management, etc.) and may support communication between various hardware, firmware, and software components.

The communication module 123 may support communication with other devices via the communication interface 110. The communication module 120 may include various software components for processing data received by the wired communication port 111 or the wireless circuit 112 of the communication interface 110.

The user interface module 123 may receive user request or input from a keyboard, a touch screen, a mouse, a microphone, etc., via the I/O interface 113, and may provide a user interface on a display.

The application 124 may include a program or module configured to be executed by one or more processors 114. Here, the application for providing information on angiography may be implemented on a server farm.

The I/O interface 113 may connect at least one of input/output devices (not illustrated) of the device 100 for providing information on hypotension, such as a display, a keyboard, a touch screen, and a microphone, to the user interface module 123. The I/O interface 113 may receive the user input (e.g., voice input, keyboard input, touch input, etc.) together with the user interface module 123, and process commands according to the received input.

The processor 114 may be connected to the communication interface 110, the memory 120, and the I/O interface 113 to control the overall operation of the device 100 for providing information on hypotension, and may perform various commands to extract data related to angiography via applications or programs stored in the memory 120.

The processor 114 may correspond to a computational device such as a central processing unit (CPU) or an application processor (AP). In addition, the processor 114 may be implemented in the form of an integrated chip (IC) such as a system on chip (SoC) in which various computational devices are integrated. Alternatively, the processor 114 may include a module for calculating an artificial intelligence (AI) machine learning model such as a neural processing unit (NPU).

Referring back to FIG. 1A, the device 100 for providing information on hypotension may receive the clinical data on the individual from the medical staff device 200 and/or the vital sign monitoring device 300, extract (generate) the first feature and the second feature from the received data, predict the hypotension of the individual based on the extracted features, and provide the predicted hypotension to the medical staff device 200 and/or the vital sign monitoring device 300.

In this way, the data provided from the device 100 for providing information on hypotension may be provided as a web page via a web browser installed in the medical staff device 200 and/or the vital sign monitoring device 300, or may be provided in the form of an application or program. In various exemplary embodiments, such data may be provided in a form included in a platform in a client-server environment.

First, the medical staff device 200 is an electronic device that requests the information on the hypotension of the individual and provides the user interface for displaying the information data (clinical data) related thereto, and may include at least one of a smart phone, a tablet personal computer (PC), a laptop, and/or a PC, etc.

More specifically, FIG. 1C illustrates a block diagram of a configuration of a medical staff device according to an exemplary embodiment of the present disclosure. The medical staff device 200 may include a memory interface 210, one or more processors 220, and a peripheral interface 230. Various components in the medical staff device 200 may be connected by one or more communication buses or signal lines.

The memory interface 210 may be connected to a memory 250 to transmit various data to the processor 220. Here, the memory 250 may include at least one type of storage media such as flash memory type, hard disk type, multimedia card micro type, card type memory (for example, SD or XD memory, etc.), RAM, SRAM, ROM, EEPROM, PROM, network storage, cloud, and blockchain database.

In various exemplary embodiments, the memory 250 may store at least one of an operating system 251, a communication module 252, a graphical user interface (GUI) module 253, a sensor processing module 254, a telephone module 255, and an application module 256. Specifically, the operating system 251 may include instructions for processing basic system services and instructions for performing hardware operations. The communication module 252 may communicate with at least one of other devices, computers, and servers. The graphical user interface (GUI) module 253 may process a graphical user interface. The sensor processing module 254 may process sensor-related functions (e.g., processing voice input received using one or more microphones 292). The telephone module 255 may process a telephone-related function. The application module 256 may perform various functions of a user application, such as electronic messaging, web browsing, media processing, navigation, imaging, and other processing functions.

In addition, the medical staff device 200 may store one or more software applications 256-1 and 256-2 associated with one type of service in the memory 250. In this case, the application 256-1 may provide information on hypotension to the medical staff device 200.

In various exemplary embodiments, the memory 250 may store a digital assistant client module 257 (hereinafter, DA client module), and accordingly, may store instructions for performing client-side functions of the digital assistant and various user data 258 (e.g., other data such as user-customized vocabulary data, preference data, user's electronic address book, to-do list, and other lists).

Meanwhile, the DA client module 257 may acquire voice input, text input, touch input, and/or gesture input of a user through various user interfaces (e.g., I/O subsystem 240) provided in the medical staff device 200.

In addition, the DA client module 257 may output data in an audiovisual or tactile form. For example, the DA client module 257 may output data composed of a combination of at least two of the following: voice, sound, notification, text message, menu, graphic, video, animation, and vibration. In addition, the DA client module 257 may communicate with a digital assistant server (not illustrated) using the communication subsystem 280.

In various exemplary embodiments, the DA client module 257 may collect additional information on the surrounding environment of the medical staff device 200 from various sensors, subsystems, and peripheral devices to construct a context associated with the user input. For example, the DA client module 257 may provide context information along with the user input to a digital assistant server to infer the user's intention. Here, the context information that may accompany the user input may include sensor information, such as lighting, ambient noise, ambient temperature, images of the surrounding environment, and videos. As another example, the context information may include physical states (e.g., device orientation, device position, device temperature, power level, speed, acceleration, motion pattern, cellular signal strength, etc.) of the medical staff device 200. As another example, the context information may include information (e.g., processes running on the medical staff device 200, installed programs, past and present network activity, background services, error logs, resource usage, etc.) related to a software status of the medical staff device 200.

In various exemplary embodiments, the memory 250 may include additional or deleted instructions, and furthermore, the medical staff device 200 may also include additional components or exclude some components in addition to the components illustrated in FIG. 1C.

The processor 220 may control the overall operation of the medical staff device 200, and may execute various commands to implement various data interfaces for angiography by running applications or programs stored in the memory 250.

The processor 220 may correspond to a computational device such as a central processing unit (CPU) or an application processor (AP). In addition, the processor 220 may be implemented in the form of an integrated chip (IC) such as a system on chip (SoC) in which various computational devices, such as a neural processing unit (NPU), are integrated.

The peripheral interface 230 may be connected to various sensors, subsystems, and peripheral devices to provide data so that the medical staff device 200 may perform various functions. Here, the functionality performed by the medical staff device 200 may be understood as being performed by the processor 220.

The peripheral interface 230 may receive data from a motion sensor 260, an illumination sensor (light sensor) 261, and a proximity sensor 262, so the medical staff device 200 may perform orientation, light, proximity detection functions, etc. As another example, the peripheral interface 230 may receive data from other sensors 263 (positioning system—GPS receiver, temperature sensor, and biometric sensor), so the medical staff device 200 may perform functions related to the other sensors 263.

In various exemplary embodiments, the medical staff device 200 may include a camera subsystem 270 connected to the peripheral interface 230 and an optical sensor 271 connected thereto, so the medical staff device 200 may perform various capturing functions, such as taking pictures and recording video clips.

In various exemplary embodiments, the medical staff device 200 may include a communication subsystem 280 connected to the peripheral interface 230. The communication subsystem 280 may include one or more wired/wireless networks and may include various communication ports, radio frequency transceivers, and optical transceivers.

In various exemplary embodiments, the medical staff device 200 may include an audio subsystem 290 connected to the peripheral interface 230, and the audio subsystem 290 may include one or more speakers 291 and one or more microphones 292, so that the medical staff device 200 may perform voice-activated functions, such as voice recognition, voice duplication, digital recording, and telephone functions.

In various exemplary embodiments, the medical staff device 200 may include the I/O subsystem 240 connected to the peripheral interface 230. For example, the I/O subsystem 240 may control a touch screen 243 included in the medical staff device 200 via a touch screen controller 241. As an example, the touch screen controller 241 may detect the user's contact and movement or the stop of the user's contact and movement using any one of a plurality of touch sensing technologies, such as a capacitive type, a resistive type, an infrared type, a surface acoustic wave technology, and a proximity sensor array. As another example, the I/O subsystem 240 may control other input/control devices 244 included in the medical staff device 200 via other input controller(s) 242. As an example, the other input controller(s) 242 may control one or more buttons, rocker switches, thumb-wheels, infrared ports, USB ports, and pointer devices, such as a stylus.

Referring back to FIG. 1A, as the medical staff device 200 includes the configuration described above, the medical staff device 200 may ultimately receive the features extracted from the clinical data related to the hypotension generated from the device 100 for providing information on hypotension, and prediction data on hypotension derived therefrom.

The vital sign monitoring device 300 may mean a medical device capable of measuring heart rate, respiration rate, body temperature, and blood pressure and evaluating human body functions.

Furthermore, the vital sign monitoring device 300 may be a device capable of providing clinical data for vital sign monitoring to the device 100 for providing information on hypotension, or displaying and adjusting the clinical data on the vital sign monitoring device 300. Accordingly, the vital sign monitoring device 300 may include, but is not limited to, a smartphone, a tablet personal computer (PC), a laptop, and/or a PC as described above, and may further include a general-purpose computer and/or a data server, etc., capable of providing data on an individual. Furthermore, the vital sign monitoring device 300 may include a DB capable of storing measured clinical monitoring data.

Meanwhile, the vital sign monitoring device 300 may include the same configuration as the medical staff device 200 described above in FIG. 1C in order to display videos and data related thereto. Accordingly, the vital sign monitoring device 300 may include an interface through which a user may directly input and provide data related to a vital sign for an individual from a keyboard, a touch screen, a mouse, a microphone, etc., and thus, receive the data related to the hypotension of the individual and provide the received data to the device 100 for providing information on hypotension and/or the medical staff device 200.

In this regard, FIG. 1D is a schematic diagram illustrating a user interface between a medical staff device and a vital sign monitoring device according to an exemplary embodiment of the present disclosure.

Referring to FIG. 1D, the medical staff device 200 and the vital sign monitoring device 300 used in the system for providing information on hypotension according to an exemplary embodiment of the present disclosure may receive various data generated and derived from the device 100 for providing information on hypotension. Accordingly, the medical staff device 200 and the vital sign monitoring device 300 may display the received data on the interface.

More specifically, the medical staff device 200 and the vital sign monitoring device 300 may be configured to display the prediction data 330 for vascular disease predicted from the device 100 for providing information on hypotension on the user interface screen (UI) (memory interface) 210.

Furthermore, not only the prediction data 330 described above, but also various data used in the vital sign monitoring device 300 may be displayed on the user interface screen (memory interface) 210. For example, various clinical data 320 related to an individual measured from the vital sign monitoring device 300 may be displayed on the user interface screen (memory interface) 210, but is not limited thereto.

Therefore, the medical staff device 200 and the vital sign monitoring device 300 may more easily acquire and confirm various data generated and predicted from the device 100 for providing information on hypotension.

In the end, the medical staff device 200 and the vital sign monitoring device 300 may receive various data generated and predicted from the device 100 for providing information on hypotension through the user interface screen (memory interface) 210, and display the received data through the user interface screen (memory interface) 210 and provide the received data to the user.

Referring back to FIG. 1A, various clinical data measured by the vital sign monitoring device 300 may be provided to the medical staff device 200 as well as the device 100 for providing information on hypotension. For example, not only the clinical data measured by the vital sign monitoring device 300, but also raw data stored in the vital sign monitoring device 300 in relation thereto may be transmitted to and utilized by the device 100 for providing information on hypotension and the medical staff device 200, but is not limited thereto.

In the end, the system for providing information on hypotension based on the device for providing information on hypotension according to an exemplary embodiment of the present disclosure includes the device 100 for providing information on hypotension, the medical staff device 200, and the vital sign monitoring device 300, and thus may receive various vital sign data and clinical data measured from the vital sign monitoring device 300, and at the same time, further receive the clinical data stored in the medical staff device 200, predict and determine various information (data) related to the hypotension of the individual based on the data received by the device 100 for providing information on hypotension, and provide various information (data) predicted (generated) from the device 100 for providing information on hypotension to the medical staff device 200 and/or the vital sign monitoring device 300. However, without being limited thereto, the operation of predicting and determining hypotension of an individual based on the received data may be performed by the medical staff device 200.

### Method for Providing Information on Hypotension According to Exemplary Embodiment of Present Disclosure

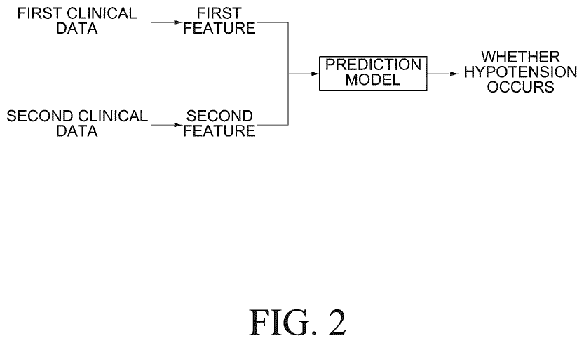

Hereinafter, a method for providing information on hypotension and its process according to an exemplary embodiment of the present disclosure will be described with reference to FIGS. 2 to 5.

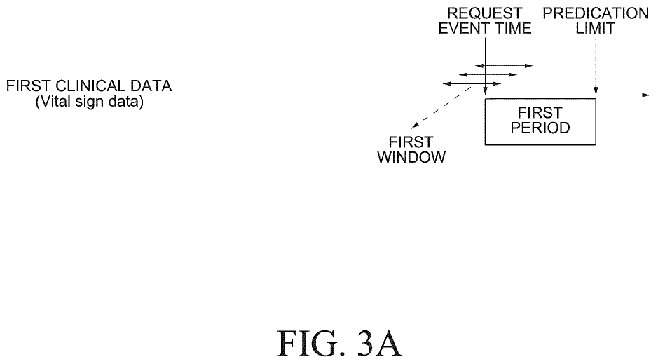

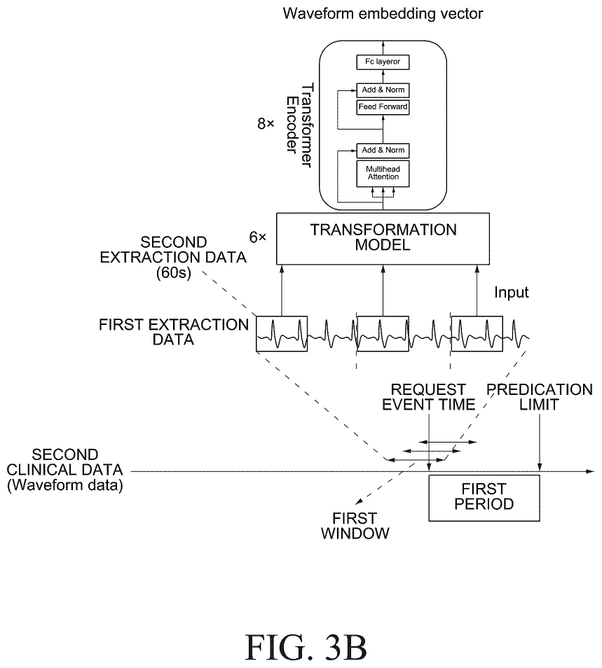

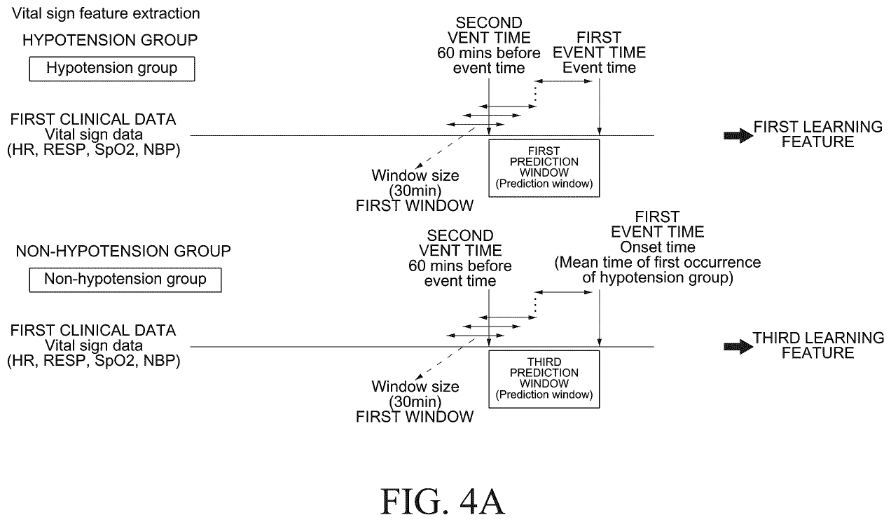

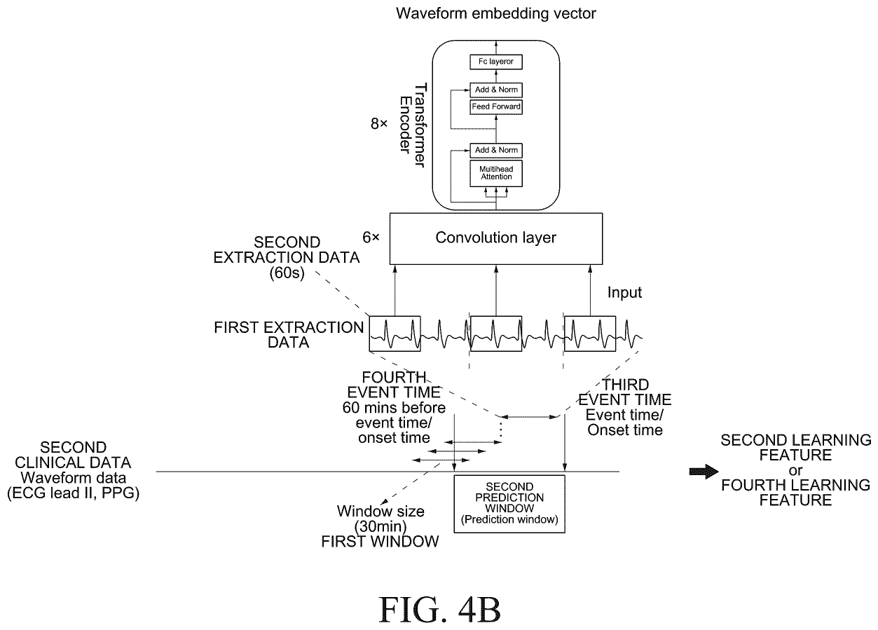

FIG. 2 is a schematic diagram illustrating a method for providing information on hypotension according to an exemplary embodiment of the present disclosure. In this case, for convenience of description, the method for providing information on hypotension will be described with reference to FIGS. 3A to 4.

Referring to FIG. 2, the method for providing information on hypotension (hereinafter, the present disclosure) according to an exemplary embodiment of the present disclosure may include first clinical data and second clinical data for an individual, and the first clinical data and the second clinical data may be received and used from a medical institution and a vital sign monitoring device. More specifically, the first clinical data is vital sign data, and may include at least one of heart rate, respiration (RESP), O2 saturation (SpO2), and non-invasive blood pressure (NBP), but is not limited thereto. Furthermore, the second clinical data is waveform data, and may include, but is not limited to, at least one of ECG lead II and photoplethysmography (PPG).

First, the method for providing information on hypotension according to an exemplary embodiment of the present disclosure may extract a statistical value for the received first clinical data as the first feature. More specifically, FIG. 3A illustrates a schematic diagram for extracting the first feature in the method for providing information on hypotension according to an exemplary embodiment of the present disclosure. As described above, the first clinical data including at least one of the heart rate (HR), the respiration (RESP), the O2 saturation (SpO2), and the non-invasive blood pressure (NBP) is vital sign data, which is time series data recorded over time. As the first clinical data of the present disclosure is the time series data, the features of the first clinical data may be extracted continuously over time. For example, the present disclosure may use a rolling window method for moving and analyzing a first clinical data by measurement time unit while maintaining a window width of a specific size for feature extraction. Accordingly, when a user requests a prediction of hypotension, a first window having a specific size is rolled windows in a time series direction, so the first feature may be extracted from the first clinical data.

In this case, the first period is a period for which the occurrence of hypotension is to be predicted, and may mean a period from a prediction request event time of a user to a specific period (prediction limit), and the prediction of the hypotension of the individual may mean whether the hypotension occurs and the probability prediction of occurrence of the hypotension for the first period. The first period may be at least one specific period of 30 to 90 minutes, but is not limited thereto as long as the first period exceeds 30 minutes. In the method for providing information on hypotension according to an exemplary embodiment of the present disclosure, the first period may preferably be 60 minutes or 50 to 60 minutes.

Furthermore, the first window may include a size for a period smaller than that of the first period. For example, in the method for providing information on hypotension according to an exemplary embodiment of the present disclosure, the first window may preferably be 30 minutes, but is not limited thereto. In addition, the first window may progress in the time series direction to continuously extract the first feature. In this case, the interval between the first windows may be 1 minute, but is not limited thereto.

Accordingly, according to the present disclosure, the method for providing information on hypotension may include extracting as the first feature the first window having a size smaller than that of the first period from the received first clinical data by the rolling windows of the first window at the predetermined time interval. For example, the method for providing information on hypotension according to an exemplary embodiment of the present disclosure may include extracting as the first feature the first window having a size of 30 minutes from the received first clinical data by the rolling windows of the first window at an interval of 1 minute. In this case, as the first window is rolled at the interval of 1 minute and the first feature is extracted, the first feature may be one or more, but is not limited thereto, and the first feature may be continuously generated at the interval of 1 minute.

Furthermore, the first feature extracted by the above-described method is a statistical value for the first clinical data, and the statistical value may include at least one of mean, variance, median, a min value, a max value, quantile, and an exponentially weighted moving average, but is not limited thereto.

Referring back to FIG. 2, the method for providing information on hypotension according to an exemplary embodiment of the present disclosure may include extracting an embedding vector for the received second clinical data as the second feature. More specifically, FIG. 3B illustrates a schematic diagram for extracting the second feature in the method for providing information on hypotension according to an exemplary embodiment of the present disclosure. The second clinical data including at least one of ECG lead II and photoplethysmography (PPG) is waveform data, which is time series data recorded over time. As the second clinical data of the present disclosure is the time series data, the feature of the second clinical data may be extracted continuously over time, and, similar to the first clinical data described above in FIG. 3A, the rolling window method may be applied. Accordingly, when a user requests a prediction of hypotension, the first window having a specific size is rolled windows in a time series direction, so the second feature may be extracted from the second clinical data.

In this case, the first period and the first window related to the second clinical data may be the same as the first period and the first window described above in FIG. 3A, but are not limited thereto.

Meanwhile, as the second clinical data of the present disclosure is waveform data, the feature extraction method may be different from that of the first clinical data. More specifically, in the present disclosure, the second clinical data may be extracted as the embedding vector. In this regard, when all the waveform data having 30 minutes extracted through the first window are generated as the embedding vector, the computational burden may increase, which may be inefficient and decrease in efficiency. Accordingly, in the present disclosure, in the case of the waveform data, as the physiological information is repeatedly measured and generated, the waveform data having a specific size extracted through the first window may be segmented, and only some of the segmented waveform data may be sampled based on one of the segmented waveform data, and then extracted as the embedding vector.

Accordingly, in the present disclosure, the first extraction data of the first window size may be extracted based on the first window from the received second clinical data.

Then, the extracted first extraction data may be sampled into a predetermined size. For example, the first extraction data may be segmented into three parts, but is not limited thereto. In this case, the first extraction data may be a size of 30 minutes when the first window is 30 minutes in size, and thus, may be segmented into three parts having a size of 10 minutes.

Next, the second extraction data for the initial partial period may be extracted from the segmented first extraction data. In this case, an initial partial period may be 60 seconds, but is not limited thereto. Accordingly, the second extraction data having a size of 60 seconds may be extracted, and this second extraction data may be selected and extracted from one of the first extraction data segmented into three parts. In the end, according to the present disclosure, it is possible to reduce the burden of the processor in the data transformation by using the second extraction data, and improve the transformation efficiency, i.e., the performance.

Then, the extracted second extraction data may be sampled. More specifically, the received waveform data is an analog signal, and thus, the data (the first extraction data and the second extraction data) extracted from the received waveform data may also be analog signals. Accordingly, in the present disclosure, the second extraction data may be sampled in order to transform the second extraction data, which is a continuous signal, into a digital form, i.e., a discrete form signal, and the sampled second extraction data may be transformed into a two-dimensional or more embedding feature in a digital form. The sampled second extraction data may have the capacity of data reduced (decreased) as the original signal is extracted at regular intervals (periodically). In the end, the present disclosure may further reduce the burden of the processor by including a low-capacity embedding feature.

Meanwhile, the sampling may be performed by a transformation model. For example, in the present disclosure, the extracted second extraction data may be input to the transformation model trained to transform the second extraction data into a two-dimensional or more embedding feature using the second extraction data as input to be transformed into a two-dimensional or more embedding feature. In this case, the transformation model is a layer that may extract features of input data through a filter and transform the extracted features, and is preferably a convolution layer, but is not limited thereto. Furthermore, the transformed two-dimensional or more embedding feature may preferably be a four-dimensional embedding feature, but is not limited thereto.

Then, the transformed embedding feature may be input to a transformer encoder and output as the embedding vector. For example, the four-dimensional embedding feature may be output (extracted) as a 12-dimensional embedding vector through the transformer encoder. In this regard, in the case of using the transformer encoder, when transforming data into the embedding, i.e., the vector, the information loss of the data may be minimized, and the data may be converted into and output as an n-dimensional embedding vector that includes (applies) an attention score. In this case, the transformer encoder may include FC layer, Add & Norm, Feed Forwar, Add & Norm, and Multihead Attention layers in that order, but is not limited thereto.

In the end, through the above-described process, the second feature in the form of the embedding vector may be extracted from the second clinical data. For example, the method for providing information on hypotension according to an exemplary embodiment of the present disclosure may include extracting the first extraction data having a size of 30 minutes from received second clinical data by the rolling windows of the first window of a size of 30 minutes at an interval of 1 minute, segmenting the extracted first extraction data having a size of 30 minutes at a size of 10 minutes so that the extracted first extraction data includes three windows, extracting the second extraction data for initial 60 seconds of one of the three windows having a size of 10 minutes, sampling the extracted second extraction data having a size of 60 seconds and transforming the sampled second extraction data into the embedding feature in the digital form, and inputting the transformed embedding feature to the transformer encoder to be output as the embedding vector.

Meanwhile, the second clinical data of the present disclosure is waveform data including ECG lead II and photoplethysmography (PPG), and frequencies of the second clinical data may be different, respectively. For example, the ECG and PPG data may be 500 Hz and 125 Hz, respectively. Accordingly, the present disclosure may further include adjusting the frequencies of the ECG and PPG data to be the same and removing noise of the ECG and PPG data. More specifically, first, in the case of the ECG data, the frequency of the ECG is down-sampled to 125 Hz, and in order to remove the noise included in the ECG, a high pass filter is applied to the ECG data to remove baseline wandering. Furthermore, a power line filter is applied to the ECG data to which the high pass is applied to further remove noise in a frequency range of 50 Hz. Furthermore, a low pass filter is applied to further remove high-frequency noise of 40 Hz or higher. Next, for the PPG data, a band pass filter is applied to the PPG data to remove noise. According to the above process, the present disclosure may provide a more reliable prediction result by removing the noise for the second clinical data.

Referring back to FIG. 2, the method for providing information on hypotension according to an exemplary embodiment of the present disclosure may extract the first feature of the statistical value and the second feature of the embedding vector from the first clinical data and the second clinical data through the process of FIGS. 3A and 3B described above.

Furthermore, the present disclosure may predict whether the hypotension occurs in the individual by inputting the extracted first feature and second feature to the prediction model. In this case, the prediction model may be a model trained to predict whether the hypotension occurs using the first feature and the second feature as inputs. For example, the prediction model of the present disclosure may be light GBM, but is not limited thereto, and as the prediction model, various models based on boosting such as XGBoost may also be used, and the prediction model may be a model based on deep learning such as CNN and DCNN, but is not limited thereto.

Furthermore, the predicted data may be data on whether the hypotension occurs by applying a function such as softmax, but is not limited thereto, and various functions and algorithms may be applied to provide various prediction values such as % for the occurrence of hypotension. Furthermore, the prediction data on the occurrence of hypotension may be data predicted within a specific period. For example, the prediction data of the present disclosure may be data that predicts when the hypotension occurs within the first period, that is, within 60 minutes from the event time for which the prediction is requested, but is not limited thereto. In addition, the hypotension in the present disclosure may mean that a systolic blood pressure (SBP) of 90 mmHg or less and a mean arterial pressure (MAP) of 60 mmHg or less occur 5 times or more for about 10 minutes.

Meanwhile, the prediction model of the present disclosure may be trained based on the received first clinical data and second clinical data. In this regard, FIG. 4A illustrates a schematic diagram for extraction of learning features from the first clinical data in the method for providing information on hypotension according to an exemplary embodiment of the present disclosure. The learning features extracted from the first clinical data of the prediction model of the present disclosure may be extracted based on the rolling window method, like the first feature and the second feature described above in FIGS. 3A and 3B, and the learning features may be extracted (output) based on a different period from the first feature and the second feature.

More specifically, the first clinical data used for training the prediction model of the present disclosure may be selected as a hypotension group or a non-hypotension group, and used for extracting the learning features. In this case, the hypotension group may mean an individual in which a systolic blood pressure (SBP) of 90 mmHg or less and a mean arterial pressure (MAP) of 60 mmHg or less occur 5 times or more for about 10 minutes, and the hypotension group may be selected based on the individual.

Therefore, the first clinical data corresponding to the hypotension group may be selected based on the above-described hypotension group criteria.

Next, a first prediction window (prediction windows) may be set from the first clinical data of the selected hypotension group. In this case, the first prediction window may be set based on a first event time and a second event time (60 minutes before the event time). More specifically, the first event time may mean an event time at which the first hypotension occurs, and may mean an event time at which a systolic blood pressure (SBP) of 90 mmHg or less and a mean arterial pressure (MAP) of 60 mmHg or less occur 5 or more times for about 10 minutes. Furthermore, the second event time may mean a specific event time before a first period from the first event time, that is, a period at which hypotension is to be predicted. For example, the second event time of the present disclosure may mean an event time that is 60 minutes before the first event time, but is not limited thereto. The first prediction window of the present disclosure may mean a window for a period from the above-described second event time to the first event time. Accordingly, the present disclosure may set a first event time for a first hypotension occurrence event time in the first clinical data of the selected hypotension group, set a second event time of a first period, that is, 60 minutes before the set first event time, and set the first clinical data for the hypotension group from the set second event time to the first event time as the first prediction window.

Then, in the set first prediction window, the first window may be rolled windows in a time series direction at a predetermined time interval to extract the first learning feature. In this case, the first window may be the window having the size smaller than that of the first period, and may be the same as the first window in FIGS. 3A and 3B. Furthermore, as the extraction of the first learning feature is extracted by applying the same rolling window method as the first feature, the statistical values for the first clinical data may be extracted as the first learning feature, and the statistical values may include at least one of the mean, the variance, the median, the min value, the max value, the quantile, and the exponentially weighted moving average.

Therefore, the extracted first learning feature may be used as training data for the prediction model, and as the first learning feature is data for the hypotension group, the prediction model of the present disclosure may train the first learning feature to predict a case including hypotension.

In addition, the present disclosure may select the first clinical data corresponding to the non-hypotension group. Next, a third prediction window (prediction windows) may be set from the first clinical data of the selected non-hypotension group. In this case, the third prediction window may be set based on the first event time and the second event time of the non-hypotension group. More specifically, the first event time in the first clinical data of the non-hypotension group may be an event time corresponding to the first event time in the hypotension group, but is not limited thereto, and may be an event time at which the first hypotension occurs. However, when the data for the hypotension group are collected from one or more individuals, the first event time in the first clinical data of the non-hypotension group may be applied with a mean value for the first event time in the hypotension group. Furthermore, the second event time in the first clinical data of the non-hypotension group may mean a specific event time before a first period from the first event time, that is, a period at which hypotension is to be predicted. For example, the second event time in the present disclosure may mean an event time that is 60 minutes before the first event time, but is not limited thereto. Similar to the first prediction window, the third prediction window of the present disclosure may mean a window for a period from the second event time to the first event time. Accordingly, the present disclosure may set a first event time for a first hypotension occurrence event time in the first clinical data of the selected non-hypotension group, set the second event time of the first period, that is, 60 minutes before the set first event time, and set the first clinical data for the non-hypotension group from the set second event time to the first event time as the third prediction window.

Then, in the set third prediction window, the first window may be rolled windows in a time series direction at a predetermined time interval to extract the third learning feature. In this case, the first window may be the window having the size smaller than that of the first period, and may be the same as the first window in FIGS. 3A and 3B. Furthermore, as the extraction of the third learning feature is extracted by applying the same rolling window method as the first feature, the statistical values for the first clinical data may be extracted as the third learning feature, and the statistical values may include at least one of the mean, the variance, the median, the min value, the max value, the quantile, and the exponentially weighted moving average.

Therefore, the extracted third learning feature may be used as training data for the prediction model, and as the third learning feature is data for the non-hypotension group, the prediction model of the present disclosure may train the third learning feature to predict a case not including hypotension.

Meanwhile, the method for providing information on hypotension according to an exemplary embodiment of the present disclosure may extract (output) the training data, that is, the learning features, from not only the first clinical data but also the second clinical data and use the extracted training data for learning.

In addition, the method for providing information on hypotension according to an exemplary embodiment of the present disclosure may derive various learning features (data) from one first clinical data as the first window is rolled windows in a time series direction at a predetermined time interval to extract the first and third learning features. Accordingly, the present disclosure may overcome limitations in collecting training data in relation to machine learning technology.

More specifically, FIG. 4B illustrates a schematic diagram for extracting the learning feature from the second clinical data in the method for providing information on hypotension according to an exemplary embodiment of the present disclosure. The learning features extracted from the second clinical data of the prediction model of the present disclosure may be extracted based on the rolling window method, like the first feature and the second feature described above in FIGS. 3A and 3B, and the learning features may be extracted (output) based on a different period from the first feature and the second feature.

More specifically, similar to the first clinical data, the second clinical data used for training the prediction model of the present disclosure may be selected as the hypotension group or the non-hypotension group, and used for extracting the learning features.

Accordingly, the second clinical data may be selected into the hypotension group and the non-hypotension group, respectively, based on the same hypotension group criteria.

For example, when the second clinical data is selected as the second clinical data of the hypotension group, the second prediction window may be set in the second clinical data of the selected hypotension group. In this case, the second prediction window may be set based on the third event time and the fourth event time of the hypotension group. More specifically, the third event time in the second clinical data of the hypotension group may be an event time corresponding to the first event time for the hypotension group in the first clinical data, but is not limited thereto, and may be an event time at which the first hypotension occurs. However, when the data for the hypotension group are collected from one or more individuals, the first event time in the second clinical data of the hypotension group may be applied with the mean value for the first event time in the hypotension group. Furthermore, the fourth event time in the second clinical data of the hypotension group may mean a specific event time before a first period from the third event time, that is, a period at which hypotension is to be predicted. For example, the fourth event time in the present disclosure may mean an event time that is 60 minutes before the third event time, but is not limited thereto. Similar to the first prediction window, the second prediction window of the present disclosure may mean a window for a period from the fourth event time to the third event time. Accordingly, the present disclosure may set the event time corresponding to the first hypotension occurrence event time or the first event time in the second clinical data of the selected hypotension group as the third event time, set the fourth event time of the first period, that is, 60 minutes before the set third event time, and set the first clinical data for the non-hypotension group from the set fourth event time to the third event time as the second prediction window.

Then, in the set second prediction window, the first window may be rolled windows in a time series direction at a predetermined time interval to extract the second learning feature. In this case, the first window may be the window having the size smaller than that of the first period, and may be the same as the first window in FIGS. 3A and 3B. Furthermore, as the second learning feature is extracted by applying the same rolling window method as the second feature, the embedding vector for the second clinical data may be extracted as the second learning feature.

Therefore, the extracted second learning feature may be used as training data for the prediction model, and as the second learning feature is data for the hypotension group, the prediction model of the present disclosure may train the second learning feature to predict the case including hypotension.

Meanwhile, the above-described process was described as the second clinical data for the hypotension group, but it may be equally applied to the non-hypotension group, so the fourth learning feature may be extracted from the second clinical data for the non-hypotension group.

Therefore, the extracted fourth learning feature may be used as training data for the prediction model, and as the fourth learning feature is data for the non-hypotension group, the prediction model of the present disclosure may train the fourth learning feature to predict the case not including hypotension.

In addition, the method for providing information on hypotension according to an exemplary embodiment of the present disclosure may derive various learning features (data) from one second clinical data as the first window is rolled windows in a time series direction at a predetermined time interval to extract the second and fourth learning features. Accordingly, the present disclosure may overcome limitations in collecting training data in relation to machine learning technology.

According to the above-described process, the present disclosure may provide a method for providing information on hypotension implemented by a processor that includes receiving the first clinical data and the second clinical data for the individual, extracting the statistical value for the received first clinical data as the first feature, extracting the embedding vector for the received second clinical data as the second feature, and predicting whether the hypotension occurs in the individual within the first period by inputting the first feature and the second feature to the prediction model trained to predict whether the hypotension occurs using the first feature and the second feature as inputs.

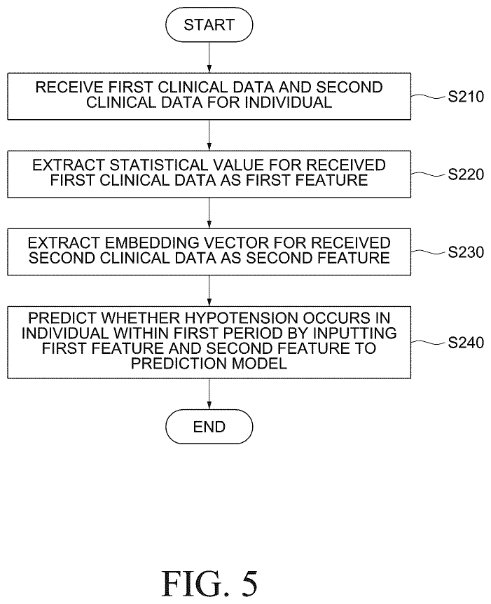

FIG. 5 is a flowchart illustrating the method for providing information on hypotension according to an exemplary embodiment of the present disclosure. In FIG. 5, the processes of FIGS. 2 to 4B described above are summarized and described.

Referring to FIG. 5, the method for providing information on hypotension according to an exemplary embodiment of the present disclosure may include receiving the first clinical data and the second clinical data for the individual (S210), extracting the statistical value for the received first clinical data as a first feature (S220), extracting the embedding vector for the received second clinical data as the second feature (S230), and predicting whether the hypotension occurs in the individual within the first period by inputting the first feature and the second feature to the prediction model trained to predict whether the hypotension occurs using the first feature and the second feature as inputs (S240). In this case, the hypotension in the present disclosure may mean that a systolic blood pressure (SBP) of 90 mmHg or less and a mean arterial pressure (MAP) of 60 mmHg or less occur 5 times or more for about 10 minutes.

First, the first clinical data received in the receiving (S210) may include at least one of the heart rate, the respiration (RESP), the O2 saturation (SpO2), and the non-invasive blood pressure (NBP), but is not limited thereto.

Furthermore, the second clinical data received in the receiving (S220) may include at least one of the ECG lead II and the photoplethysmography (PPG), but is not limited thereto.

Then, in the extracting of the statistical value as the first feature (S220), the statistical value may include at least one of the mean, the variance, the median, the min value, the max value, the quantile, and the exponentially weighted moving average, but is not limited thereto.

Furthermore, the extracting of the statistical value as the first feature (S220) may include extracting as the first feature the first window having a size smaller than that of a first period from the received first clinical data by the rolling windows of the first window at the predetermined time interval. For example, the extracting of the statistical value as the first feature (S220) may include extracting as the first feature a first window having a size of 30 minutes from the received first clinical data by the rolling windows of the first window at an interval of 1 minute. In this case, as the first feature is extracted at an interval of 1 minute, the hypotension prediction based on the first feature may be continuously performed.

Furthermore, the extracting of the embedding vector as the second feature (S230) may include extracting as the second feature the first window having the size smaller than that of the first period from the received second clinical data by the rolling windows of the first window at the predetermined time interval, and may be similar to the extracting of the first window as the first feature described above. However, since the second clinical data includes repetitive signals, when all of the second clinical data are used, the computation burden may increase, and the data efficiency may be reduced. Therefore, in the present disclosure, only a representative specific part of the repetitive signals may be extracted and the data efficiency may be improved by sampling the extracted part. Therefore, the extracting of the embedding vector as the second feature (S230) may include extracting the first extraction data having a size of the first window based on the first window from the received second clinical data, segmenting the extracted first extraction data into a predetermined size, extracting the second extraction data for a portion of an initial period from the segmented first extraction data, sampling the extracted second extraction data so that the extracted second extraction data is transformed into the embedding feature in the digital form, and inputting the embedding feature to the transformer encoder and outputting the embedding feature as the embedding vector.

In this case, the sampling step may be performed by applying an algorithm (model). For example, in the present disclosure, the sampling step may include inputting the extracted second extraction data to the transformation model trained to transform the second extraction data into the two-dimensional or more embedding feature using the second extraction data as input to transform the extracted second extraction data into the two-dimensional or more embedding feature. In this case, the transformation model may include a convolutional neural network (CNN) layer, but is not limited thereto.

Then, in the predicting step (S240), the prediction model may be trained to have the high accuracy prediction ability based on various training data derived from the first clinical data and the second clinical data received in the receiving step (S210). More specifically, according to the present disclosure, the method may further include prior to the extracting, selecting the hypotension group from the received first clinical data and second clinical data, setting the first prediction window of the first period in the first clinical data of the selected hypotension group, setting the second prediction window of the first period in the second clinical data of the selected hypotension group, extracting the first learning feature and the second learning feature from each of the set first prediction window and second prediction window by the rolling windows of the first window having the size smaller than that of the first period at the predetermined time interval, and training the prediction model based on the extracted first learning feature and second learning feature.

In this case, the setting of the first prediction window may further include setting the first event time for the first hypotension occurrence event time in the first clinical data of the selected hypotension group, setting the second event time before the first period from the first event time, and setting the first clinical data for the hypotension group from the second event time to the first event time as the first prediction window.

In addition, the setting of the second prediction window may include setting the event time corresponding to the first event time in the second clinical data of the selected hypotension group as the third event time, setting the fourth event time before the first period from the third event time, and setting the second clinical data for the non-hypotension group from the fourth event time to the third event time as the second prediction window.

Meanwhile, the first learning feature and the second learning feature described above may be the training data based on the hypotension group, and the prediction model may analyze and train when the hypotension occurs based on the training data. However, the present disclosure also derives the training data based on the non-hypotension group, and the prediction model may analyze and train when the hypotension does not occur based on the derived training data.

Accordingly, according to the present disclosure, the method may further include, prior to the extracting, selecting the non-hypotension group from the received first clinical data and second clinical data, setting the third prediction window of the first period in the first clinical data of the selected non-hypotension group, setting the fourth prediction window of the first period in the second clinical data of the selected non-hypotension group, extracting the third learning feature and the fourth learning feature from each of the set third prediction window and fourth prediction window by the rolling windows of the first window having the size smaller than that of the first period at the predetermined time interval, and training the prediction model based on the extracted third learning feature and fourth learning feature.

In this case, the setting of the third prediction window may further include setting the first event time for the first hypotension occurrence event time in the first clinical data of the selected non-hypotension group, setting the second event time before the first period from the first event time, and setting the first clinical data for the non-hypotension group from the second event time to the first event time as the third prediction window.

In addition, the setting of the fourth prediction window may include setting the event time corresponding to the first event time in the second clinical data of the selected non-hypotension group as the third event time, setting the fourth event time before the first period from the third event time, and setting the second clinical data for the non-hypotension group from the fourth event time to the third event time as the fourth prediction window.

According to the above process, the method for providing information on hypotension according to an exemplary embodiment of the present disclosure may continuously predict the risk of occurrence of hypotension occurring within a specific period (60 minutes) in the future by receiving the clinical data in real time. In addition, the present disclosure may further include the second clinical data as well as the first clinical data and have the high level of prediction accuracy by simultaneously using the first and second clinical data for prediction.

### Verification of Method for Providing Information on Hypotension According to Exemplary Embodiment of Present Disclosure

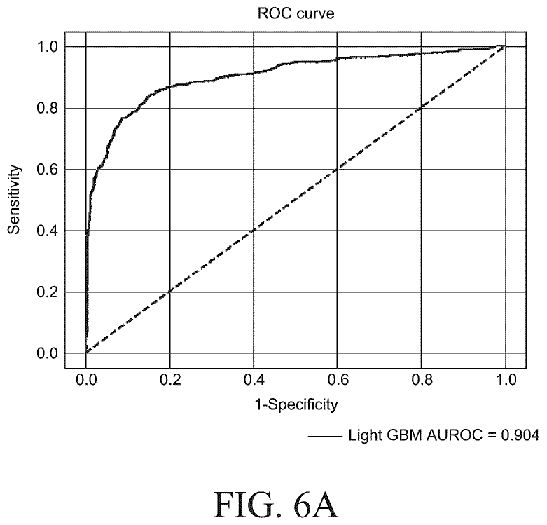

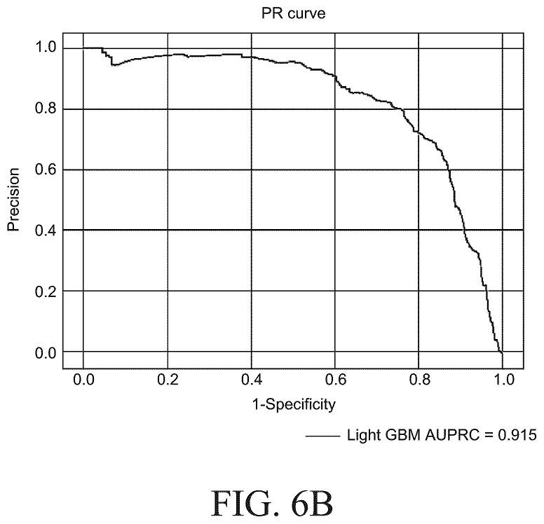

Hereinafter, the performance of the method for providing information on hypotension according to an exemplary embodiment of the present disclosure will be verified with reference to FIGS. 6A to 9. In this case, as the training and verification data used in FIGS. 6A to 9, clinical data of patients hospitalized in the intensive care unit of Yongin Severance Hospital between Jan. 1, 2021 and Jun. 30, 2022 was used, and data corresponding to exclusion criteria (minors, patients with no measured numerical vital sign, patients whose data length was less than an average hypotension occurrence event time of 3,348 minutes, patients without simultaneous ECG and PPG among waveform vital signs, patients with pacemakers attached) was excluded from among the 3,382 data hospitalized in the intensive care unit of Yongin Severance Hospital during the entire period, so 938 clinical data were used for the training and verification of the present disclosure. Furthermore, in the training and verification process of the present disclosure, for one level, data having a length of 30 minutes starting from 1 hour and 29 minutes before the label reference time is extracted with a difference of 1 minute, so a total of 60 training data were generated for each patient. Accordingly, the training cohort contained 39,960 data, and the test cohort contained 16,320 data. Furthermore, when training the model with a training cohort, 36,000 data were distributed to the training group at each fold with 10-fold cross validation, and 3,960 data were randomly distributed to the validation group. A light GBM model, which is the prediction model of the present disclosure, was trained based on the above-described data, and the performance of the light GBM model was evaluated.

FIG. 6A is a diagram illustrating an AUROC curve result for the method for providing information on hypotension according to an exemplary embodiment of the present disclosure.

Referring to FIG. 6a, the AUROC for the prediction model of the method for providing information on hypotension according to an exemplary embodiment of the present disclosure is 0.904, which may mean that the prediction model has high sensitivity and specificity in predicting the hypotension, that is, excellent performance (0.8 or higher based on the AUROC is considered excellent).

FIG. 6B is a diagram illustrating an AUPRC curve result for the method for providing information on hypotension according to an exemplary embodiment of the present disclosure.

Referring to FIG. 6B, the AUPRC for the prediction model of the method for providing information on hypotension according to an exemplary embodiment of the present disclosure is 0.915, which may mean that the prediction model has high precision and reproducibility in predicting the hypotension, that is, excellent performance.

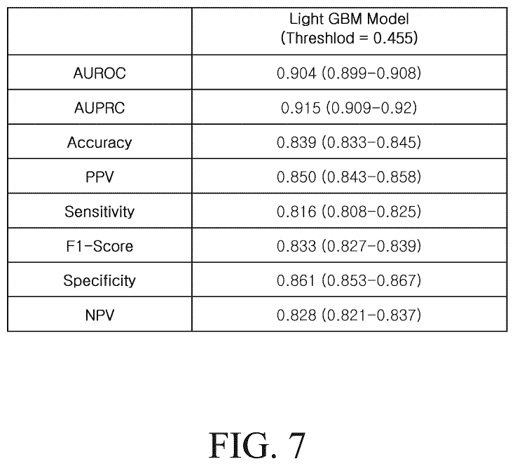

In addition, FIG. 7 illustrates the performance evaluation result for the prediction model of the method for providing information on hypotension according to an exemplary embodiment of the present disclosure. In this case, a 95% confidence interval (CI) was presented after 500 bootstrap resamples for a test set.

When the threshold value of 0.455, at which Youden J index of the model is maximized, is applied to the test set, the AUROC, AUPRC, accuracy, positive predictive value (PPV), sensitivity, F1-score, specificity, and negative predictive value (NPV) are 0.904, 0.915, 0.839, 0.850, 0.816, 0.833, 0.861, and 0.828, respectively, that are all greater than or equal to 0.8, which may mean that the prediction model of the present disclosure has excellent performance.

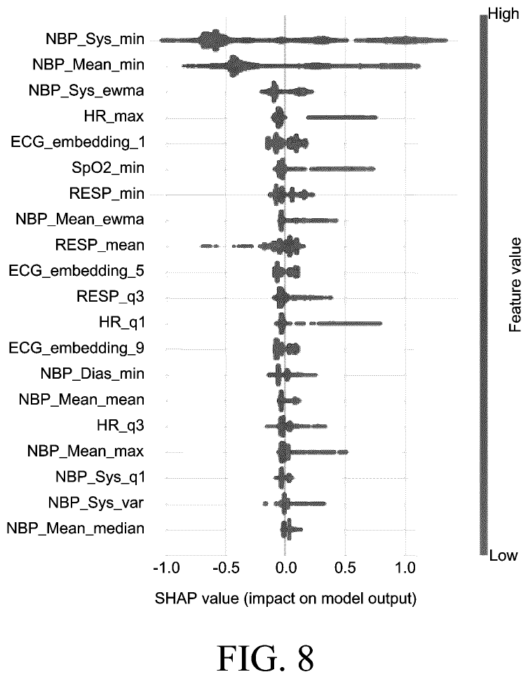

FIG. 8 is a diagram illustrating a SHAP value result for the method for providing information on hypotension according to an exemplary embodiment of the present disclosure. In this case, the features for the clinical data of the present disclosure used for the SHAP value evaluation are as shown in [Table 1] below.

TABLE 1

| Vital | | |
|---|---|---|
| sign | | |
| variable | Feature name | Description |
| HR | HR_mean | Mean value of heart rate |
| | HR_medain | Median value of heart rate |
| | HR variance | Variance value of heart rate |
| | HR_min | Min value of heart rate |
| | HR_max | Max value of heart rate |
| | HR_q1 | 1st quartile value of heart rate |
| | HR_q3 | 3rd quartile value of heart rate |
| | HR_ewma | Ewma value of heart rate |
| RESP | RESP_mean | Mean value of respiratory rate |
| | RESP_medain | Median value of respiratory rate |
| | RESP_variance | Variance value of respiratory rate |
| | RESP_min | Min value of respiratory rate |
| | RESP_max | Max value of respiratory rate |
| | RESP_q1 | 1st quartile value of respiratory rate |
| | RESP_q3 | 3rd quartile value of respiratory rate |
| | RESP_ewma | Ewma value of respiratory rate |
| Sp02 | SpO2_mean | Mean value of oxygen saturation |
| | SpO2_medain | Median value of oxygen saturation |
| | SpO2 variance | Variance value of oxygen saturation |
| | SpO2_min | Min value of oxygen saturation |
| | SpO2_max | Max value of oxygen saturation |
| | SpO2_q1 | 1st quartile value of oxygen saturation |
| | SpO2_q3 | 3rd quartile value of oxygen saturation |
| | SpO2_ewma | Ewma value of oxygen saturation |
| NBP_Sys | NBP_Sys_mean | Mean value of systolic blood pressure |
| | | measured by non-invasive BP |
| | NBP_Sys_medain | Median value of systolic blood pressure |
| | | measured by non-invasive BP |
| | NBP_Sys_variance | Variance value of systolic blood pressure |
| | | measured by non-invasive BP |
| | NBP_Sys_min | Min value of systolic blood pressure |
| | | measured by non-invasive BP |
| | NBP Sys_max | Max value of systolic blood pressure |
| | | measured by non-invasive BP |
| | NBP_Sys_q1 | 1st quartile value of systolic blood pressure |
| | | measured bynon-invasive BP |
| | NBP_Sys_q3 | 3rd quartile value of systolic blood pressure |
| | | measured by non-invasive BP |
| | NBP Sys_ewma | Ewma value of systolic blood pressure |
| | | measured by non-invasive BP |
| NBP_Dias | NBP_Dias_mean | Mean value of diastolic blood pressure |
| | | measured by non-invasive BP |
| | NBP_Dias_mcdain | Median value of diastolic blood pressure |
| | | measured by non-invasive BP |
| | NBP_Dias_variance | Variance value of diastolic blood pressure |
| | | measured by non-invasive BP |
| | NBP_Dias_min | Min value of diastolic blood pressure |
| | | measured by non- invasive BP |
| | NBP_Dias_max | Max value of diastolic blood pressure |
| | | measured by non-invasive BP |
| | NBP_Dias_q1 | 1st quartile value of diastolic blood |
| | | pressure measured by non-invasive BP |
| | NBP Dias q3 | 3rd quartile value of diastolic blood pressure |
| | | measured by non-invasive BP |
| | NBP Dias cwma | Ewma value of diastolic blood pressure |
| | | measured by non-invasive BP |
| NBP | NBP Mean mean | Mean value of mean blood pressure |
| Mean | | measured by non-invasive BP |
| | NBP_Mean_medain | Median value of mean blood pressure |
| | | measured by non-invasive BP |
| | NBP_Mean_variance | Variance value of mean blood pressure |
| | | measured by non-invasive BP |
| | NBP_Mean_min | Min value of mean blood pressure |
| | | measured by non-invasive BP |
| | NBP_Mean_max | Max value of mean blood pressure |
| | | measured by non-invasive BP |
| | NBP_Mean_q1 | 1st quartile value of mean blood pressure |
| | | measured by non-invasive BP |
| | NBP_Mean_q3 | 3rd quartile value of mean blood pressure |
| | | measured by non-invasive BP |
| | NBP Mean ewma | Ewma value of mean blood pressure |
| | | measured by non-invasive BP |
| ECG | ECG_embedding_1 | First 10-minute segment's 1-dimension |
| | | of the ECG embedding |
| | ECG_embedding_2 | First 10-minute segment's 2-dimension |
| | | of the ECG embedding |
| | ECG_embedding_3 | First 10-minute segment's 3-dimension |
| | | of the ECG embedding |
| | ECG_embedding_4 | First 10-minute segment's 4-dimension |
| | | of the ECG embedding |
| | ECG_embedding_5 | Second 10-minute segment's 1-dimension |
| | | of the ECG embedding |
| | ECG_embedding_6 | Second 10-minute segment's 2-dimension |
| | | of the ECG embedding |
| | ECG_embedding_7 | Second 10-minute segment's 3-dimension |
| | | of the ECG embedding |
| | ECG_embedding_8 | Second 10-minute segment's 4-dimension |
| | | of the ECG embedding |
| | ECG_embedding_9 | Third 10-minute segment's 1-dimension |
| | | of the ECG embedding |
| | ECG_embedding_10 | Third 10-minute segment's 2-dimension |
| | | of the ECG embedding |
| | ECG_embedding_11 | Third 10-minute segment's 3-dimension |
| | | of the ECG embedding |
| | ECG_embedding_12 | Third 10-minute segment's 4-dimension |
| | | of the ECG embedding |
| PPG | PPG_embedding_1 | First 10-minute segment's 1-dimension |
| | | of the PPG embedding |
| | PPG_embedding_2 | First 10-minute segment's 2-dimension |
| | | of the PPG embedding |
| | PPG_embedding_3 | First 10-minute segment's 3-dimension |
| | | of the PPG embedding |
| | PPG_embedding_4 | First 10-minute segment's 4-dimension |
| | | of the PPG embedding |
| | PPG_embedding_5 | Second 10-minute segment's 1-dimension |
| | | of the PPG embedding |
| | PPG_embedding_6 | Second 10-minute segment's 2-dimension |
| | | of the PPG embedding |
| | PPG_embedding_7 | Second 10-minute segment's 3-dimension |
| | | of the PPG embedding |
| | PPG_embedding_8 | Second 10-minute segment's 4-dimension |
| | | of the PPG embedding |
| | PPG_embedding_9 | Third 10-minute segment's 1-dimension |
| | | of the PPG embedding |
| | PPG_embedding_10 | Third 10-minute segment's 2-dimension |
| | | of the PPG embedding |
| | PPG_embedding_11 | Third 10-minute segment's 3-dimension |
| | | of the PPG embedding |
| | PPG_embedding_12 | Third 10-minute segment's 4-dimension |
| | | of the PPG embedding |

Referring to FIG. 8, the result of analyzing the feature importance of features derived from the clinical data of the present disclosure using the SHAP value shows that the features for the second clinical data (features embedding ECG data) have a high influence on the prediction of hypotension, which means that the second clinical data is an important factor in the prediction of hypotension.

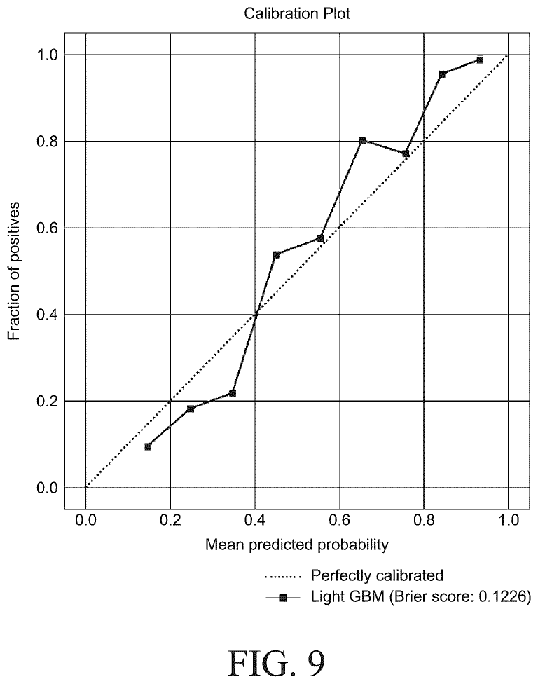

FIG. 9 is a consistency evaluation result for a method for providing information on hypotension according to an exemplary embodiment of the present disclosure. In this case, a calibration plot was used to evaluate the consistency between the predicted probability of the prediction model of the present disclosure and the actual occurrence probability.

Referring to FIG. 9, a Brier score is 0.1226, which may mean that the probability prediction value of the prediction model is accurate (the closer the Brier score is to the minimum value of 0, the more accurate it is).

In other words, it may mean that the prediction model of the present disclosure has high consistency with the actual occurrence probability and has high reliability in the prediction.

Although the exemplary embodiments of the present disclosure have been described in more detail with reference to the accompanying drawings, the present disclosure is not necessarily limited to these exemplary embodiments, but may be variously modified without departing from the scope and spirit of the present disclosure. Accordingly, the exemplary embodiments disclosed in the present disclosure and the accompanying drawings are used not to limit but to describe the spirit of the present disclosure. The scope of the present disclosure is not limited only to the exemplary embodiments and the accompanying drawings. Therefore, it is to be understood that the exemplary embodiments described above are illustrative rather than being restrictive in all aspects. The scope of the present disclosure should be interpreted by the following claims, and it should be interpreted that all spirits equivalent to the following claims fall within the scope of the present disclosure.

What is claimed is:

1. A method for providing information on hypotension implemented by a processor, comprising:
receiving first clinical data and second clinical data for an individual;
extracting a statistical value for the received first clinical data as a first feature;
extracting an embedding vector for the received second clinical data as a second feature; and
predicting whether the hypotension occurs in the individual within a first period by inputting the first feature and the second feature to a prediction model trained to predict whether the hypotension occurs using the first feature and the second feature as inputs.

2. The method of claim 1, wherein the hypotension has a systolic blood pressure (SBP) of 90 mmHg or less and a mean arterial pressure (MAP) of 60 mmHg or less, which occur 5 times or more for about 10 minutes.

3. The method of claim 1, wherein the first clinical data includes at least one of heart rate, respiration (RESP), O2 saturation (SpO2), and non-invasive blood pressure (NBP), the second clinical data includes at least one of ECG lead II and photoplethysmography (PPG), and
the statistical value includes at least one of mean, variance, median, a min value, a max value, quantile, and an exponentially weighted moving average.

4. The method of claim 1, wherein the extracting of the statistical value as the first feature includes extracting as the first feature a first window having a size smaller than that of the first period from the received first clinical data by time-series interval movement (rolling windows) of the first window at a predetermined time interval.

5. The method of claim 1, wherein the extracting of the embedding vector as the second feature includes extracting as the second feature a first window having the size smaller than that of the first period from the received second clinical data by the rolling windows of the first window at the predetermined time interval, and
the extracting of the embedding vector as the second feature includes:
extracting first extraction data having a size of the first window based on the first window from the received second clinical data;
segmenting the extracted first extraction data into a predetermined size;
extracting second extraction data for a portion of an initial period from the segmented first extraction data;
sampling the extracted second extraction data so that the extracted second extraction data is transformed into an embedding feature in a digital form; and
inputting the embedding feature to a transformer encoder and outputting the embedding feature as the embedding vector.

6. The method of claim 5, wherein the sampling further includes transforming the second extraction data into a two-dimensional or more embedding feature by inputting the extracted second extraction data to a transformation model trained to transform the second extraction data into the two-dimensional or more embedding feature using the second extraction data as input.

7. The method of claim 1, further comprising:
prior to the extracting,
selecting a hypotension group from the received first clinical data and second clinical data;
setting a first prediction window of the first period in the first clinical data of the selected hypotension group;
setting a second prediction window of the first period in the second clinical data of the selected hypotension group;
extracting a first learning feature and a second learning feature from each of the set first prediction window and second prediction window by rolling windows of a first window having a size smaller than that of the first period at a predetermined time interval; and
training the prediction model based on the extracted first learning feature and second learning feature, and
the setting of the first prediction window includes:
setting a first event time for a first hypotension occurrence event time in the first clinical data of the selected hypotension group;
setting a second event time before the first period from the first event time; and
setting the first clinical data for the hypotension group from the second event time to the first event time as the first prediction window, and
the setting of the second prediction window includes:
setting an event time corresponding to the first event time in the second clinical data of the selected hypotension group as a third event time;
setting a fourth event time before the first period from the third event time; and
setting the second clinical data for a non-hypotension group from the fourth event time to the third event time as the second prediction window.

8. The method of claim 1, further comprising:
prior to the extracting,
selecting a non-hypotension group from the received first clinical data and second clinical data;
setting a third prediction window of the first period in the first clinical data of the selected non-hypotension group;
setting a fourth prediction window of the first period in the second clinical data of the selected non-hypotension group;
extracting a third learning feature and a fourth learning feature from each of the set third prediction window and fourth prediction window by rolling windows of a first window having a size smaller than that of the first period at a predetermined time interval; and
training the prediction model based on the extracted third learning feature and fourth learning feature.

9. The method of claim 8, wherein the setting of the third prediction window includes:
setting a first event time for a first hypotension occurrence event time in the first clinical data of the selected non-hypotension group;
setting a second event time before the first period from the first event time; and
setting the first clinical data for the non-hypotension group from the second event time to the first event time as the third prediction window.

10. The method of claim 9, wherein the setting of the fourth prediction window includes:
setting an event time corresponding to the first event time in the second clinical data of the selected non-hypotension group as a third event time;
setting a fourth event time before the first period from the third event time; and
setting the second clinical data for the non-hypotension group from the fourth event time to the third event time as the fourth prediction window.

11. A device for providing information on hypotension, comprising:
a communication unit configured to receive first clinical data and second clinical data for an individual; and
a processor configured to communicate with the communication unit,
wherein the processor is configured to extract a statistical value for the received first clinical data as a first feature;
extract an embedding vector for the received second clinical data as a second feature; and
predict whether the hypotension occurs in the individual within a first period by inputting the first feature and the second feature to a prediction model trained to predict whether the hypotension occurs using the first feature and the second feature as input.

12. The device of claim 11, wherein the hypotension has a systolic blood pressure (SBP) of 90 mmHg or less and a mean arterial pressure (MAP) of 60 mmHg or less which occur 5 times or more for about 10 minutes.

13. The device of claim 11, wherein the first clinical data includes at least one of heart rate, respiration (RESP), O2 saturation (SpO2), and non-invasive blood pressure (NBP),
the second clinical data includes at least one of ECG lead II and photoplethysmography (PPG), and
the statistical value includes at least one of mean, variance, median, a mini value, a max value, quantile, and an exponentially weighted moving average.

14. The device of claim 11, wherein the processor is configured to extract as the first feature a first window having a size smaller than that of the first period from the received first clinical data by rolling windows of the first window at a predetermined time interval.

15. The device of claim 11, wherein the processor is configured to extract as the second feature a first window having a size smaller than that of the first period from the received second clinical data by rolling windows of the first window at a predetermined time interval,
extract first extraction data having a size of the first window based on the first window from the received second clinical data,
segment the extracted first extraction data into a predetermined size,
extract second extraction data for a portion of an initial period from the segmented first extraction data,
sample the extracted second extraction data so that the extracted second extraction data is transformed into an embedding feature in a digital form, and
input the embedding feature to a transformer encoder and output the embedding feature as the embedding vector.

16. The device of claim 15, wherein the processor is further configured to transform the second extraction data into a two-dimensional or more embedding feature by inputting the extracted second extraction data to a transformation model trained to transform the second extraction data into the two-dimensional or more embedding feature using the second extraction data as input.

17. The device of claim 11, wherein the processor is further configured to select a hypotension group from the received first clinical data and second clinical data,
set a first prediction window of the first period in the first clinical data of the selected hypotension group,
set a second prediction window of the first period in the second clinical data of the selected hypotension group,
extract a first learning feature and a second learning feature from each of the set first prediction window and second prediction window by rolling windows of a first window having a size smaller than that of the first period at a predetermined time interval, and
train the prediction model based on the extracted first learning feature and second learning feature, and
the processor is configured to set a first event time for a first hypotension occurrence event time in the first clinical data of the selected hypotension group,
set a second event time before the first period from the first event time,
set the first clinical data for the hypotension group from the second event time to the first event time as the first prediction window,
set an event time corresponding to the first event time in the second clinical data of the selected hypotension group as a third event time,
set a fourth event time before the first period from the third event time, and
set the second clinical data for a non-hypotension group from the fourth event time to the third event time as the second prediction window.

18. The device of claim 11, wherein the processor is further configured to select a non-hypotension group from the received first clinical data and second clinical data,
set a third prediction window of the first period in the first clinical data of the selected non-hypotension group,
set a fourth prediction window of the first period in the second clinical data of the selected non-hypotension group,
extract a third learning feature and a fourth learning feature from each of the set third prediction window and fourth prediction window by rolling windows of a first window having a size smaller than that of the first period at a predetermined time interval, and
train the prediction model based on the extracted third learning feature and fourth learning feature.

19. The device of claim 18, wherein the processor is configured to set a first event time for a first hypotension occurrence event time in the first clinical data of the selected non-hypotension group,
set a second event time before the first period from the first event time, and
set the first clinical data for the non-hypotension group from the second event time to the first event time as the third prediction window.

20. The device of claim 19, wherein the processor is configured to set an event time corresponding to the first event time in the second clinical data of the selected non-hypotension group as a third event time,
set a fourth event time before the first period from the third event time, and
set the second clinical data for the non-hypotension group from the fourth event time to the third event time as the fourth prediction window.
